SEQUENCE LISTING (1 sequence)

SEQ ID NO: 1 - DNA, 7262 bp, Homo sapiens
ccagcccaagtttttagggtggggatccagactggttatacgtaccttcagtccttctcccagaggaaggcagaaacacctcaaagcctgcatgtaagaacatctactgagaaattattttaatcagacaccagctgagtgggagaaagaaaaagaacagagaagaacaaacaaaactcccttggtcttggatgtaagagaatccagcagagatggactggagtttcttcagagtagttgcaatgctgttcatttttctggtggtggtagaagttaacagtgaattccgaatccaggtaagagattataacactaaaaatggcaccatcaaatggcattcaatccgaaggcagaaacgtgaatggatcaagttcgcagcagcctgtcgtgaaggtgaagacaactcaaagaggaacccaatcgccaaaattcactcagattgtgctgcaaaccagcaagttacataccgcatctctggagtaggaattgatcagccaccatatgggatctttgtcattaatcagaaaactggtgaaattaatataacatccatagttgatcgagaggtcactcctttcttcattatctactgccgagctctgaactcaatgggccaagatttagagaggcctctagagctcagagtcagggttttggatataaatgacaaccctccagtgttttcaatggctacatttgcaggacaaatagaagaaaattctaatgcaaatacactggtgatgatactcaatgctactgacgcagatgaaccgaacaatttgaactcaaaaatagccttcaagattataagacaagaaccttcagattcaccaatgtttattatcaacagaaatactggagaaattcgaacgatgaataattttctagacagagagcaatacggccagtatgctcttgctgtaagaggctctgaccgagatggcggggcagatggcatgtcagcggaatgtgagtgcaacattaaaatcctcgatgtcaatgataatatcccttacatggaacagtcttcatataccatagaaattcaagaaaatactctaaattcaaatttgctcgagattagagtaattgatttggatgaagagttctcagctaactggatggcagtaattttctttatctctggaaatgaaggaaattggtttgagatagaaatgaatgaaagaacaaatgtgggaattttaaaggttgttaagcccttagattatgaagctatgcagagtctgcaactcagtattggtgtcagaaataaagctgaatttcatcattcaattatgtctcaatataaactgaaagcatctgcaatttctgtgactgtgttaaatgtaattgaaggcccagtgtttcgtccaggttcaaagacatatgttgtaactggtaatatgggatcaaatgataaagtgggagactttgtagctactgacctggacacaggtagaccttcaacgactgttaggtatgtaatgggaaataatccagctgacctgctagctgttgattcaagaacaggcaaactcactttgaaaaataaagttaccaaggaacagtacaatatgctcggaggaaaataccaaggaacgattctctctatagatgataatcttcaaagaacttgcactggtacaattaatattaacattcaaagttttggtaatgacgacaggactaatacagagccgaacactaaaattactaccaatactggcagacaagaaagtacttcttccactaactatgataccagcacaacttctactgactctagccaagtatattcttctgaacccggaaacggagccaaagatttgttatcagacaatgtacattttggtcctgctggcattggactcctcatcatgggattcttggtcttaggattggtcccatttttgatgatctgttgtgattgtggaggtgctcctcgtagtgcagctggctttgagcctgttcccgaatgttcagatggagcaattcattcatgggcagtagaaggaccacagcctgaacccagggatataaccactgtcataccacaaataccacctgataacgcaaatataattgaatgcattgacaactcaggagtttatacaaatgagtatggtggcagagaaatgcaagatctgggaggaggagagagaatgacaggatttgaactaacagagggagttaaaacttcaggaatgcctgagatatgtcaagaatactctggaacattaagaagaaattctatgagggaatgtagagaaggaggtctgaatatgaatttcatggaaagctacttctgtcagaaagcatatgcttacgcagatgaagatgaaggacgcccatctaatgactgtttgctcatatatgacatcgaaggtgtaggttcccctgctggctctgtgggttgttgtagcttcattggagaagacctggatgacagcttcttggataccctgggacctaaatttaagaagttggcagacatcagcctaggaaaagaatcatatccagaccttgatccttcttggccaccacaaagcactgaaccagtttgccttcctcaggaaacagagcccgttgttagtggacacccaccaatctccccacatttcggcactaccacagtaatttctgagagcacctatccctcgggacctggtgtactgcatcctaagcctattctcgatcctctgggctatggtaatgtcactgtgaccgagtcttacaccacctctgacactctgaagccctctgtgcacgttcacgataaccgaccagcatcaaacgtggtagtgacagagagagtggtcggcccaatctctggcgctgatttgcatggaatgttagagatgcctgacttgcgagatgggtcgaatgttatagtgacagaaagggtaatagcaccaagctctagtctacccacctctctgactatccatcatcctagagagtcttcaaatgtggtagtgacagaaagagtaatccaaccaacttccggcatgataggtagtctgagtatgcaccccgagttagccaatgcccacaatgtcattgtgacagagagggttgtttctggtgctggcgtaactggaattagtggcaccactgggatcagcggtggcataggcagcagtggcctggttggcaccagcatgggtgctgggagcggtgccctgagtggagctggcataagtggtggtggcattggcctgagcagcttgggagggacagccagcattggccacatgaggagttcctctgaccatcactttaaccaaaccattgggtccgcctcccctagcacagctcgaagtcgaatcacaaagtatagtaccgtgcaatatagcaagtagtcaggaccccagctcactttttcatagtcattgtggtttagatccaattcccaccactaaaaaaccaacaatgtgatttataacgcacaacttcgtgctcaggtcatctaggagcaaggtgagaaatcacaatgagaaaaataaatggaaacaccactgctaggggagagctctccttagcattcataaacttttctcttatattaggactaaggaactaaaacttgaggcagagtcttctttgtgcctgagtggcctgtagtccatctccagcatgtaactggccttacgatggcaattggcatcattctccttgctctgttttgcttttccatatagctcgagcaaaattcaaaaagaactaaatatgcaatatatgttcatatctatgggaaaaatctaaaatgtgtgccagatgccctgttggtttcacagataacataaataaaaattcaaccacagatttatacaagggttaaccattttttttaagtttgactacatagtcaagtccacaagccatcaagcactcctaccttaattattgcactagagaaaataaattccaaattaggaagtgtttcctaggaggaaaattccattagagagtggcaataggatgaggtttcttcagggtaaactagcaatgcctgagcctgaaccttaatgtggggcctcagttaaatctcctgtggagtcaaggattcttctgattctagtgtgtgtttagtgatagatgtagtcttgacgaatattgcttactggtgaggttgaggaatatcacactcgtctttccctttaccactgtggttttgacttaagaaagcaaaactcactaagtttacttctcgaattgaagcaagtgaggcctgacatggttgtcatcactagtggcaaatgaccttccaagtaagcagatgggaactgaattgtgttttcaggttttgtttttagtaggtgatattcattcgtatccagctctttattacatagctctgaagttaaaatgatttacataggccgagctgtggacaaaaaaaaaagaagcagcagcttgtagtatgcttaagctttggggaatttttttttaaggggatctaaaaaaatgtttttagaacatgtaaaatgtttaatggtgaaagttggaaaagaattcttctgtaaagtaataccatgctaattattcgcttttagtaagtaaagtagtggttgctttagcaaacctctgctgccattttgcaggaatcaaccaggaacctttagcagaattgacaatatggtgttgataagcatgaaataataatagaaacctattctgctagtttatctcaccctctaatttttctcactagcataaattttaaattcctgatttgatttgtcaataagatcttggcttatatatgctgatttataggtagtgtccaaattatatagtataacatatttttctagtttcaaaatttagtaatgtcctatttatgatatatcatttctgtgtgtttgctatgtagtattacccaattaaaaatctctaaaaagaattaaagcattctaagaaaaaggtaaatttactattgcatggtacagaaattttttctttcttaaatacaatgttactataagctcactaaaatgaaactctatatgacaaaataaaattagaaaaaaatttgccctggagttgtgaattatatacaacttttaaagaatttaccccaattactcaaatttcccaggaaattacaaagccaaagaatattcaacttcctccactggtcaaaagaggataggagtgaattactgaacctagagctattttgctctgtaacaacagataaggctaatattttaaaagccacagtatacatcttcttttaactctgtagaatatgtaaaattttgatagtctgtagtatgctaaatgcagaagtataaataaagtcattcaaagggagtcttttctttttctgacacttagggggccacattaaggatgggtaatctttccaggaataaagtcaaaaggtatttattaagacatacttgagtatgcctgggtccaggagttttaaggaatagaaataagtattaatgaattaattaagtaatttattaaagggaatggtagctgaccacaggaaacttgcttactgttttgatatgaaatatcatcacaagcttttcttaagacatctgatatcttccagagatattttttaggttgtcttgcaaacaacaaaatcactgtctttaataactgttgctgtcaaaatccattggttgttaagatccccccaatttagttacatctgaactcctaaacactgttaaacgatgggaaaaacaagaaaaaacatggccatttgagtcattgagtcatctatctttctaggaagatactttctaaccaaacttttcttccaggattgcaaattgatgggaaaaacaagaaaaactgaagtattagtcacctatctttctgggaagatactttccaactaattttttcttccaggattgcacactgattttccatttagtcctaaattttaaaattcccttttcaagacatcaacgattttagtagttatttaaaggcatgtcatttttcaatgaagaagttttgggcagaacttcattcttcttcttagatgtttactctagatcatatacatcatgtcatagaccaagaagagatatggaaattattttataagtgaatactataattaggattcaagctgagtttcagatcaacttgctcttaacaaaaggaaaaagaaatagtaatttaatactatgtatgtatggtttgaaaacaaaccacaatgtttataaaatatctatctgactgtctaaagaggtaatctttaggagcaaaaatcagtgtattataaatactttaccatttaatatcaaccaaaataccatctcaagctaattttgacactgaattacagatatatctgctacatattatttacttctaagcatgttgtctgatgtaattgcatttgcactgaaaaattaaaagaaaaagtacatatttagggttatttatatatcttcatctagacatctgttctacatttgtgtataaagtttttagcatcataatttttattcaagaaaatgttctgacaaaattttaattatatgtcttcaaaaattacattttttactctagtaagtagatgtttttagttatctggcaatttatttctgaatttataccaatgtttgattgtcatggtacaaaatatatgacaccctttaacttttgctggagttgaaaggcattataatctttagcataaatggccatgactattttggaaagacatttaagacccaaagcaaacttttaaaagtatttgccacattttcccatgcctatttcataaattccaacttttttttttacaatttctggatttttaagacccatttcacattgcactaggatacagcagtccacagtagagtgctactctccttgaaatcaaatctgtcttccacttccggattattcaatttatgttaggacaaatcttgactagatcaacctgttttccatcagataattttaaaacaatgtgtaatcttgtttgtctacattctctccccagtttagctgtatttgaattactaaatgctttatcgtcaaactgtacctagtctaacttatttttcttttgctgtcgttttacaagcattttaaaattctaatattcatctctggtggtgtttaacacaaggttctcttattcaagtttcaatataaaagtttttggattatttgggtgctagtttcttgcttggttatctgttcgtttttttaagttgatttgtaatttccaaagagttatgcatacagcaataaaattattaatatgc

(19) United States
(12) Patent Application Publication
(10) Pub. No.: US 2019/0125826 A1
(43) Pub. Date: May 2, 2019

(54) METHODS AND PHARMACEUTICAL COMPOSITION FOR THE TREATMENT OF INFLAMMATORY SKIN DISEASES ASSOCIATED WITH DESMOGLEIN-1 DEFICIENCY

(71) Applicants: INSERM (INSTITUT NATIONAL DE LA SANTÉ ET DE LA MÉDICALE), Paris (FR); ASSISTANCE PUBLIQUE-HÔPITAUX DE PARIS (APHP), Paris (FR); UNIVERSITÉ PARIS DESCARTES, Paris (FR); FONDATION IMAGINE, Paris (FR)

(72) Inventors: Christine BODEMER, Paris (FR); Asma SMAHI, Paris (FR); Elodie BAL, Paris (FR); Laura POLIVKA, Paris (FR); Smail HADJ-RABIA, Paris (FR)

(21) Appl. No.: 16/095,504

(22) PCT Filed: Apr. 21, 2017
(86) PCT No.: PCT/EP2017/059467

§ 371 Date: Oct. 22, 2018

(30) Foreign Application Priority Data
Apr. 22, 2016 (FR) 16305470.3

(51) Int. Cl. A61K 38/17; A61P 17/00; C12Q 1/6883

(52) U.S. Cl. CPC A61K 38/177; A61P 17/00; C12Q 2600/106; C12Q 2600/156; C12Q 1/6883

(57) ABSTRACT

The present invention relates to methods and pharmaceutical composition for the treatment of inflammatory skin diseases associated with desmoglein-1 deficiency. The inventors show, for the first time, that the structural protein DSG1 directly acts as a novel and unexpected inhibitor of epithelial inflammation via the inhibition of NF-κB signaling pathway. In particular, the present invention relates to a method of treating an inflammatory skin disease associated with desmoglein-1 deficiency in a subject in need thereof comprising administering to the subject a therapeutically effective amount of an agent capable of restoring the expression of desmogelin-1. Particularly, the inventors carried out the whole exome sequencing, histopathological, electron microscopy, immunofluorescence and immunological analyses in two unrelated patients presenting with SAMEC syndrome.

### FIELD OF THE INVENTION

The present invention relates to methods and pharmaceutical composition for the treatment of inflammatory skin diseases associated with desmoglein-1 deficiency.

### BACKGROUND OF THE INVENTION

The epidermis constitutes a physical and functional barrier against environmental agents, essential in maintaining skin homeostasis. The combination of inflammation and barrier dysfunction is evident in the pathogenesis of severe dermatitis such as atopic dermatitis.^{1,2} However little is known about how epithelial barrier dysfunction and immunological dysregulation interact and contribute to the initiation and maintenance of such inflammatory diseases. Recently, mutations in desmoplakin (DSP) and desmoglein-1 (DSG1) genes have been involved in an inherited inflammatory skin disease characterized by Severe dermatitis, multiple Allergies and Metabolic wasting (SAM syndrome, MIM#603165).³⁻⁶ These two genes encode two structural components of desmosomes critical for intercellular junctions and maintenance of epithelial barrier integrity. Desmosomes are particularly abundant in epidermis, digestive epithelium and heart. All proteins constituting the desmosomal complex scaffolding are present in the epidermis, while only some of them are present in the heart, such as DSP but not DSG1.⁷

### SUMMARY OF THE INVENTION

The present invention relates to methods and pharmaceutical composition for the treatment of inflammatory skin diseases associated with desmoglein-1 deficiency. In particular, the present invention is defined by the claims.

### DETAILED DESCRIPTION OF THE INVENTION

Loss of epidermal integrity is known to play a role in the pathogenesis of inflammatory disorders, especially those associating allergic manifestations. However the intertwined mechanisms of epithelial barrier dysfunction and immunological dysregulation should be clarified. The inventors decipher the relationship between epithelial barrier disruption and immunological dysregulation, in a rare disorder combining Severe dermatitis, multiple Allergies, Metabolic wasting, (SAM syndrome) with Ectodermal dysplasia and arrhythmogenic Cardiomyopathy (hereby called SAMEC syndrome). Whole exome sequencing, histopathological, electron microscopy, immunofluorescence and immunological analyses were performed in two unrelated patients presenting with SAMEC syndrome. SAMEC syndrome is due to a heterozygous mutation in DSP gene coding for desmoplakin. The DSP mutations, identified here, induce the deficiency of desmoglein-1 (DSG1), a close partner of DSP. Both DSP and DSG1 are two structural proteins involved in epithelial barrier integrity via desmosomes. The inventors show, for the first time, that the structural protein DSG1 directly acts as a novel and unexpected inhibitor of epithelial inflammation via the inhibition of NF-κB signaling pathway. By deciphering SAMEC (SAM, Ectodermal dysplasia and arrhythmogenic Cardiomyopathy) syndrome, the inventors show that the structural protein DSG1 is a new inhibitor of NF-κB-mediated inflammation in the skin. DSG1 deficiency observed in patients with atopic dermatitis, Netherton, SAM, and SAMEC syndromes could play a crucial role in epithelial inflammation.

Accordingly a first object of the present invention relates to a method of treating an inflammatory skin disease associated with desmoglein-1 deficiency in a subject in need thereof comprising administering to the subject a therapeutically effective amount of an agent capable of restoring the expression of desmogelin-1.

A second object of the present invention relates to a method of treating an inflammatory skin disease associated with desmoglein-1 deficiency in a subject in need thereof comprising administering to the subject a therapeutically effective amount of an inhibitor of NF-κB signaling pathway.

A third object of the present invention relates to a method of treating an inflammatory skin disease associated with desmoglein-1 deficiency in a subject in need thereof comprising administering to the subject a therapeutically effective amount of an inhibitor of at least one cytokine selected from the group consisting of IL-6, IL-8, IL-1beta and TSLP.

As used herein, the term “inflammatory skin disease” refers to diseases characterized by occurrence of a skin lesion resulting from infiltration of inflammatory cells such as activated helper T cells and monocytes. According to the present invention, inflammatory skin diseases comprise in particular dermatitis such as atopic dermatitis, Netherton syndrome, SAM, and SAMEC syndromes. As used herein the term “atopic dermatitis” has its general meaning in the art and refers to a chronic disease affecting the skin. Atopic dermatitis is produced by a combination of genetic and environmental factors and associated with excessive IgE antibody formation. As used herein the term “Netherton syndrome” has its general meaning in the art and refers to a rare autosomal recessive genodermatosis caused by mutations in SPINK5 (LEKTI) one of the major inhibitor of the skin kallikrein cascade.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein the term “desmoglein-1” or “DSG1” has its general meaning in the art and refers to a member of the desmoglein protein subfamily. DSG1 is also known as DSG; CDHF4; EPKHE; PPKS1; SPPK1; EPKHIA. An exemplary human nucleic acid sequence of DSG1 is represented by SEQ ID NO:1.

| SEQ ID NO: 1 | |
|---|---|
| 1 | ccagcccaag tttttagggt ggggatccag actggttata cgtaccttca gtccttctcc |
| 61 | cagaggaagg cagaaacacc tcaaagcctg catgtaagaa catctactga gaaattattt |
| 121 | taatcagaca ccagctgagt gggagaaaga aaaagaacag agaagaacaa acaaaactcc |
| 181 | cttggtcttg gatgtaagag aatccagcag agatggactg gagtttcttc agagtagttg |
| 241 | caatgctgtt catttttctg gtggtggtag aagttaacag tgaattccga atccaggtaa |
| 301 | gagattataa cactaaaaat ggcaccatca aatggcattc aatccgaagg cagaaacgtg |
| 361 | aatggatcaa gttcgcagca gcctgtcgtg aaggtgaaga caactcaaag aggaacccaa |
| 421 | tcgccaaaat tcactcagat tgtgctgcaa accagcaagt tacataccgc atctctggag |
| 481 | taggaattga tcagccacca tatgggatct ttgtcattaa tcagaaaact ggtgaaatta |
| 541 | atataacatc catagttgat cgagaggtca ctcctttctt cattatctac tgccgagctc |
| 601 | tgaactcaat gggccaagat ttagagaggc ctctagagct cagagtcagg gttttggata |
| 661 | taaatgacaa ccctccagtg ttttcaatgg ctacatttgc aggacaaata gaagaaaatt |
| 721 | ctaatgcaaa tacactggtg atgatactca atgctactga cgcagatgaa ccgaacaatt |
| 781 | tgaactcaaa aatagccttc aagattataa gacaagaacc ttcagattca ccaatgttta |
| 841 | ttatcaacag aaatactgga gaaattcgaa cgatgaataa ttttctagac agagagcaat |
| 901 | acggccagta tgctcttgct gtaagaggct ctgaccgaga tggcggggca gatggcatgt |
| 961 | cagcggaatg tgagtgcaac attaaaatcc tcgatgtcaa tgataatatc ccttacatgg |
| 1021 | aacagtcttc atataccata gaaattcaag aaaatactct aaattcaaat ttgctcgaga |
| 1081 | ttagagtaat tgatttggat gaagagttct cagctaactg gatggcagta attttcttta |
| 1141 | tctctggaaa tgaaggaaat tggtttgaga tagaaatgaa tgaaagaaca aatgtgggaa |
| 1201 | ttttaaaggt tgttaagccc ttagattatg aagctatgca gagtctgcaa ctcagtattg |
| 1261 | gtgtcagaaa taaagctgaa tttcatcatt caattatgtc tcaatataaa ctgaaagcat |
| 1321 | ctgcaatttc tgtgactgtg ttaaatgtaa ttgaaggccc agtgtttcgt ccaggttcaa |
| 1381 | agacatatgt tgtaactggt aatatgggat caaatgataa agtgggagac tttgtagcta |
| 1441 | ctgacctgga cacaggtaga ccttcaacga ctgttaggta tgtaatggga aataatccag |
| 1501 | ctgacctgct agctgttgat tcaagaacag gcaaactcac tttgaaaaat aaagttacca |
| 1561 | aggaacagta caatatgctc ggaggaaaat accaaggaac gattctctct atagatgata |
| 1621 | atcttcaaag aacttgcact ggtacaatta atattaacat tcaaagtttt ggtaatgacg |
| 1681 | acaggactaa tacagagccg aacactaaaa ttactaccaa tactggcaga caagaaagta |
| 1741 | cttcttccac taactatgat accagcacaa cttctactga ctctagccaa gtatattctt |
| 1801 | ctgaacccgg aaacggagcc aaagatttgt tatcagacaa tgtacatttt ggtcctgctg |
| 1861 | gcattggact cctcatcatg ggattcttgg tcttaggatt ggtcccattt ttgatgatct |
| 1921 | gttgtgattg tggaggtgct cctcgtagtg cagctggctt tgagcctgtt cccgaatgtt |
| 1981 | cagatggagc aattcattca tgggcagtag aaggaccaca gcctgaaccc agggatataa |
| 2041 | ccactgtcat accacaaata ccacctgata acgcaaatat aattgaatgc attgacaact |
| 2101 | caggagttta tacaaatgag tatggtggca gagaaatgca agatctggga ggaggagaga |
| 2161 | gaatgacagg atttgaacta acagagggag ttaaaacttc aggaatgcct gagatatgtc |
| 2221 | aagaatactc tggaacatta agaagaaatt ctatgaggga atgtagagaa ggaggtctga |
| 2281 | atatgaattt catggaaagc tacttctgtc agaaagcata tgcttacgca gatgaagatg |
| 2341 | aaggacgccc atctaatgac tgtttgctca tatatgacat cgaaggtgta ggttcccctg |
| 2401 | ctggctctgt gggttgttgt agcttcattg gagaagacct ggatgacagc ttcttggata |
| 2461 | ccctgggacc taaatttaag aagttggcag acatcagcct aggaaaagaa tcatatccag |
| 2521 | accttgatcc ttcttggcca ccacaaagca ctgaaccagt ttgccttcct caggaaacag |
| 2581 | agcccgttgt tagtggacac ccaccaatct ccccacattt cggcactacc acagtaattt |
| 2641 | ctgagagcac ctatccctcg ggacctggtg tactgcatcc taagcctatt ctcgatcctc |
| 2701 | tgggctatgg taatgtcact gtgaccgagt cttacaccac ctctgacact ctgaagccct |
| 2761 | ctgtgcacgt tcacgataac cgaccagcat caaacgtggt agtgacagag agagtggtcg |
| 2821 | gcccaatctc tggcgctgat ttgcatggaa tgttagagat gcctgacttg cgagatgggt |
| 2881 | cgaatgttat agtgacagaa agggtaatag caccaagctc tagtctaccc acctctctga |
| 2941 | ctatccatca tcctagagag tcttcaaatg tggtagtgac agaaagagta atccaaccaa |
| 3001 | cttccggcat gataggtagt ctgagtatgc accccgagtt agccaatgcc cacaatgtca |
| 3061 | ttgtgacaga gagggttgtt tctggtgctg gcgtaactgg aattagtggc accactggga |
| 3121 | tcagcggtgg cataggcagc agtggcctgg ttggcaccag catgggtgct gggagcggtg |
| 3181 | ccctgagtgg agctggcata agtggtggtg gcattggcct gagcagcttg ggagggacag |
| 3241 | ccagcattgg ccacatgagg agttcctctg accatcactt taaccaaacc attgggtccg |
| 3301 | cctcccctag cacagctcga agtcgaatca caaagtatag taccgtgcaa tatagcaagt |
| 3361 | agtcaggacc ccagctcact ttttcatagt cattgtggtt tagatccaat tcccaccact |
| 3421 | aaaaaaccaa caatgtgatt tataacgcac aacttcgtgc tcaggtcatc taggagcaag |
| 3481 | gtgagaaatc acaatgagaa aaataaatgg aaacaccact gctaggggag agctctcctt |
| 3541 | agcattcata aacttttctc ttatattagg actaaggaac taaaacttga ggcagagtct |
| 3601 | tctttgtgcc tgagtggcct gtagtccatc tccagcatgt aactggcctt acgatggcaa |
| 3661 | ttggcatcat tctccttgct ctgttttgct tttccatata gctcgagcaa aattcaaaaa |
| 3721 | gaactaaata tgcaatatat gttcatatct atgggaaaaa tctaaaatgt gtgccagatg |
| 3781 | ccctgttggt ttcacagata acataaataa aaattcaacc acagatttat acaagggtta |
| 3841 | accatttttt ttaagtttga ctacatagtc aagtccacaa gccatcaagc actcctacct |
| 3901 | taattattgc actagagaaa ataaattcca aattaggaag tgtttcctag gaggaaaatt |
| 3961 | ccattagaga gtggcaatag gatgaggttt cttcagggta aactagcaat gcctgagcct |
| 4021 | gaaccttaat gtggggcctc agttaaatct cctgtggagt caaggattct tctgattcta |
| 4081 | gtgtgtgttt agtgatagat gtagtcttga cgaatattgc ttactggtga ggttgaggaa |
| 4141 | tatcacactc gtctttccct ttaccactgt ggttttgact taagaaagca aaactcacta |
| 4201 | agtttacttc tcgaattgaa gcaagtgagg cctgacatgg ttgtcatcac tagtggcaaa |
| 4261 | tgaccttcca agtaagcaga tgggaactga attgtgtttt caggttttgt ttttagtagg |
| 4321 | tgatattcat tcgtatccag ctctttatta catagctctg aagttaaaat gatttacata |
| 4381 | ggccgagctg tggacaaaaa aaaaagaagc agcagcttgt agtatgctta agctttgggg |
| 4441 | aatttttttt taaggggatc taaaaaaatg tttttagaac atgtaaaatg tttaatggtg |
| 4501 | aaagttggaa aagaattctt ctgtaaagta ataccatgct aattattcgc ttttagtaag |
| 4561 | taaagtagtg gttgctttag caaacctctg ctgccatttt gcaggaatca accaggaacc |
| 4621 | tttagcagaa ttgacaatat ggtgttgata agcatgaaat aataatagaa acctattctg |
| 4681 | ctagtttatc tcaccctcta atttttctca ctagcataaa ttttaaattc ctgatttgat |
| 4741 | ttgtcaataa gatcttggct tatatatgct gatttatagg tagtgtccaa attatatagt |
| 4801 | ataacatatt tttctagttt caaaatttag taatgtccta tttatgatat atcatttctg |
| 4861 | tgtgtttgct atgtagtatt acccaattaa aaatctctaa aaagaattaa agcattctaa |
| 4921 | gaaaaaggta aatttactat tgcatggtac agaaattttt tctttcttaa atacaatgtt |
| 4981 | actataagct cactaaaatg aaactctata tgacaaaata aaattagaaa aaaatttgcc |
| 5041 | ctggagttgt gaattatata caacttttaa agaatttacc ccaattactc aaatttccca |
| 5101 | ggaaattaca aagccaaaga atattcaact tcctccactg gtcaaaagag gataggagtg |
| 5161 | aattactgaa cctagagcta ttttgctctg taacaacaga taaggctaat attttaaaag |
| 5221 | ccacagtata catcttcttt taactctgta gaatatgtaa aattttgata gtctgtagta |
| 5281 | tgctaaatgc agaagtataa ataaagtcat tcaaagggag tcttttcttt ttctgacact |
| 5341 | tagggggcca cattaaggat gggtaatctt tccaggaata aagtcaaaag gtatttatta |
| 5401 | agacatactt gagtatgcct gggtccagga gttttaagga atagaaataa gtattaatga |
| 5461 | attaattaag taatttatta aagggaatgg tagctgacca caggaaactt gcttactgtt |
| 5521 | ttgatatgaa atatcatcac aagcttttct taagacatct gatatcttcc agagatattt |
| 5581 | tttaggttgt cttgcaaaca acaaaatcac tgtctttaat aactgttgct gtcaaaatcc |
| 5641 | attggttgtt aagatccccc caatttagtt acatctgaac tcctaaacac tgttaaacga |
| 5701 | tgggaaaaac aagaaaaaac atggccattt gagtcattga gtcatctatc tttctaggaa |
| 5761 | gatactttct aaccaaactt ttcttccagg attgcaaatt gatgggaaaa acaagaaaaa |
| 5821 | ctgaagtatt agtcacctat ctttctggga agatactttc caactaattt tttcttccag |
| 5881 | gattgcacac tgattttcca tttagtccta aattttaaaa ttcccttttc aagacatcaa |
| 5941 | cgattttagt agttatttaa aggcatgtca tttttcaatg aagaagtttt gggcagaact |
| 6001 | tcattcttct tcttagatgt ttactctaga tcatatacat catgtcatag accaagaaga |
| 6061 | gatatggaaa ttattttata agtgaatact ataattagga ttcaagctga gtttcagatc |
| 6121 | aacttgctct taacaaaagg aaaaagaaat agtaatttaa tactatgtat gtatggtttg |
| 6181 | aaaacaaacc acaatgttta taaaatatct atctgactgt ctaaagaggt aatctttagg |
| 6241 | agcaaaaatc agtgtattat aaatacttta ccatttaata tcaaccaaaa taccatctca |
| 6301 | agctaatttt gacactgaat tacagatata tctgctacat attatttact tctaagcatg |
| 6361 | ttgtctgatg taattgcatt tgcactgaaa aattaaaaga aaaagtacat atttagggtt |
| 6421 | atttatatat cttcatctag acatctgttc tacatttgtg tataaagttt ttagcatcat |
| 6481 | aatttttatt caagaaaatg ttctgacaaa attttaatta tatgtcttca aaaattacat |
| 6541 | tttttactct agtaagtaga tgtttttagt tatctggcaa tttatttctg aatttatacc |
| 6601 | aatgtttgat tgtcatggta caaaatatat gacacccttt aacttttgct ggagttgaaa |
| 6661 | ggcattataa tctttagcat aaatggccat gactattttg gaaagacatt taagacccaa |
| 6721 | agcaaacttt taaaagtatt tgccacattt tcccatgcct atttcataaa ttccaacttt |
| 6781 | tttttttaca atttctggat ttttaagacc catttcacat tgcactagga tacagcagtc |
| 6841 | cacagtagag tgctactctc cttgaaatca aatctgtctt ccacttccgg attattcaat |
| 6901 | ttatgttagg acaaatcttg actagatcaa cctgttttcc atcagataat tttaaaacaa |
| 6961 | tgtgtaatct tgtttgtcta cattctctcc ccagtttagc tgtatttgaa ttactaaatg |
| 7021 | ctttatcgtc aaactgtacc tagtctaact tatttttctt ttgctgtcgt tttacaagca |
| 7081 | ttttaaaatt ctaatattca tctctggtgg tgtttaacac aaggttctct tattcaagtt |
| 7141 | tcaatataaa agtttttgga ttatttgggt gctagtttct tgcttggtta tctgttcgtt |
| 7201 | tttttaagtt gatttgtaat ttccaaagag ttatgcatac agcaataaaa ttattaatat |
| 7261 | gc |

As used herein, the term “DSG1 deficiency” denotes that the cells of the subject or a part thereof have a DSG1 dysfunction, a low or a null expression of desmoglein-1. Said deficiency may typically result from a mutation in so that the pre-ARN m is degraded through the NMD (non sense mediated decay) system. Said deficiency may also typically result from a mutation so that the protein is misfolded and degraded through the proteasome. Said deficiency may also result from a loss of function mutation leading to a dysfunction of the protein. Said deficiency may also result from an epigenetic control of gene expression (e.g. methylation) so that the gene is less expressed in the cells of the subject. Said deficiency may also result from a repression of the DSG1 gene induce by a particular signalling pathway

Accordingly, in one embodiment, the methods of treatment of the present invention comprise a first step for determining whether the subject suffering from an inflammatory skin disease has a DSG1 deficiency.

In some embodiments, the first step consists in detecting the mutation that is responsible for the DSG1 deficiency. In some embodiments, the mutation is selected from table A. In some embodiments, the presence of the mutation selected from the group consisting of c.A1757C/p.H586P, c.T1828C/p.S610P may be searched for. One skilled in the art can easily identify a mutation in DSG1 gene.

TABLE A
mutations responsible for a DSG1 deficiency

| Gene | Mutation | Codon | Proteine | Maladie | Base |
|---|---|---|---|---|---|
| DSG1 | CGA/TGA | 26 | Arg/X | Striate PPK | HGMD |
| DSG1 | TCA/TAA | 132 | Ser/X | Striate PPK | HGMD |
| DSG1 | AGA/TGA | 144 | Arg/X | Striate PPK | HGMD |
| DSG1 | c.515C > T | | | PPK | Dua-Awereh et al |
| DSG1 | CAA/TAA | 201 | Gln/X | Striate PPK | HGMD |
| DSG1 | CGA/TGA | 219 | Arg/X | Striate PPK | HGMD |
| DSG1 | GGT/GGC | 244 | Gly/Gly | Pemphigus | HGMD |
| DSG1 | TAT/TAA | 365 | Tyr/X | Striate PPK | HGMD |
| DSG1 | IVS2 as-1 G/A | | | Striate PPK | HGMD |
| DSG1 | IVS4 as-2 A/G | | | Striate PPK | HGMD |
| DSG1 | IVS5 ds-3 C/T | | | Striate PPK | HGMD |
| DSG1 | IVS9 as-3 C/G | | | Striate PPK | HGMD |
| DSG1 | IVS11 as-1 G/T | | | Striate PPK | HGMD |
| DSG1 | 396delA | | | Striate PPK | HGMD |
| DSG1 | 466delA | | | Striate PPK | HGMD |
| DSG1 | 542delA | | | Striate PPK | HGMD |
| DSG1 | 643delA | | | Striate PPK | HGMD |
| DSG1 | ins40T | 121 | | Focal PPK | HGMD |
| DSG1 | ins359C | 1079 | | Striate PPK | HGMD |
| DSG1 | c.49-1G > A | | | SAM | E Sprecher |
| DSG1 | c.1861delG | | | SAM | E Sprecher |
| DSG1 | c.2659C > T | | p.R887* | SAM | C Has |
| DSG1 | c.2614delA | | p.Ile872Serfs*10 | SAM | J Fischer |

Typically the mutation may be detected by analyzing a DSG1 polynucleotide. In the context of the invention, DSG1 polynucleotides include mRNA, genomic DNA and cDNA derived from mRNA. DNA or RNA can be single stranded or double stranded. These may be utilized for detection by amplification and/or hybridization with a probe, for instance. The nucleic acid sample may be obtained from any cell source or tissue biopsy. Non-limiting examples of cell sources available include without limitation blood cells, buccal cells, epithelial cells, fibroblasts, or any cells present in a tissue obtained by biopsy. Cells may also be obtained from body fluids, such as blood, plasma, serum, lymph, etc. DNA may be extracted using any methods known in the art, such as described in Sambrook et al, 1989. R A may also be isolated, for instance from tissue biopsy, using standard methods well known to the one skilled in the art such as guanidium thiocyanate-phenol-chloroform extraction. DSG1 mutations may be detected in a RNA or DNA sample, preferably after amplification. For instance, the isolated RNA may be subjected to coupled reverse transcription and amplification, such as reverse transcription and amplification by polymerase chain reaction (RT-PCR), using specific oligonucleotide primers that are specific for a mutated site or that enable amplification of a region containing the mutated site. According to a first alternative, conditions for primer annealing may be chosen to ensure specific reverse transcription (where appropriate) and amplification; so that the appearance of an amplification product be a diagnostic of the presence of a particular DSG1 mutation. Otherwise, RNA may be reverse-transcribed and amplified, or DNA may be amplified, after which a mutated site may be detected in the amplified sequence by hybridization with a suitable probe or by direct sequencing, or any other appropriate method known in the art. For instance, a cDNA obtained from RNA may be cloned and sequenced to identify a mutation in DSG1 sequence. Actually numerous strategies for genotype analysis are available (Antonarakis et al, 1989; Cooper et al, 1991; Grompe, 1993). Briefly, the polynucleotide may be tested for the presence or absence of a restriction site. When a base substitution mutation creates or abolishes the recognition site of a restriction enzyme, this allows a simple direct PCR test for the mutation. Further strategies include, but are not limited to, direct sequencing, restriction fragment length polymorphism (RFLP) analysis; hybridization with allele-specific oligonucleotides (ASO) that are short synthetic probes which hybridize only to a perfectly matched sequence under suitably stringent hybridization conditions; allele-specific PCR; PCR using mutagenic primers; ligase-PCR, HOT cleavage; denaturing gradient gel electrophoresis (DGGE), temperature denaturing gradient gel electrophoresis (TGGE), single-stranded conformational polymorphism (SSCP) and denaturing high performance liquid chromatography (Kuklin et al, 1997). Direct sequencing may be accomplished by any method, including without limitation chemical sequencing, using the Maxam-Gilbert method; by enzymatic sequencing, using the Sanger method; mass spectrometry sequencing; sequencing using a chip-based technology; and real-time quantitative PCR. Preferably, DNA from a subject is first subjected to amplification by polymerase chain reaction (PCR) using specific amplification primers. However several other methods are available, allowing DNA to be studied independently of PCR, such as the rolling circle amplification (RCA), the Invader™assay, or oligonucleotide ligation assay (OLA). OLA may be used for revealing base substitution mutations. According to this method, two oligonucleotides are constructed that hybridize to adjacent sequences in the target nucleic acid, with the join sited at the position of the mutation. DNA ligase will covalently join the two oligonucleotides only if they are perfectly hybridized. Therefore, useful polynucleotides, in particular oligonucleotide probes or primers, according to the present invention include those which specifically hybridize the regions where the mutations are located. Oligonucleotide probes or primers may contain at least 10, 15, 20 or 30 nucleotides. Their length may be shorter than 400, 300, 200 or 100 nucleotides.

The mutation may be also detected at a protein level (e.g. for loss of function mutation) according to any appropriate method known in the art. In particular a biological sample, such as a tissue biopsy, obtained from a subject may be contacted with antibodies specific of a mutated form of DSG1 protein, i.e. antibodies that are capable of distinguishing between a mutated form of DSG1 and the wild-type protein, to determine the presence or absence of a DSG1 specified by the antibody. The antibodies may be monoclonal or polyclonal antibodies, single chain or double chain, chimeric antibodies, humanized antibodies, or portions of an immunoglobulin molecule, including those portions known in the art as antigen binding fragments Fab, Fab′, F(ab′)2 and F(v). They can also be immunoconjugated, e.g. with a toxin, or labelled antibodies. Whereas polyclonal antibodies may be used, monoclonal antibodies are preferred for they are more reproducible in the long run. Procedures for raising “polyclonal antibodies” are also well known. Alternatively, binding agents other than antibodies may be used for the purpose of the invention. These may be for instance aptamers, which are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by Exponential enrichment (SELEX) of a random sequence library.

In some embodiments, the DSG1 deficiency is detected by determining the expression level of DSG1. In some embodiment, the DSG1 expression level may be determined by any well known method in the art. In particular, an immunohistochemistry (IHC) method may be preferred. IHC specifically provides a method of detecting targets in a sample or tissue specimen in situ. The overall cellular integrity of the sample is maintained in IHC, thus allowing detection of both the presence and location of the targets of interest. Typically a sample is fixed with formalin, embedded in paraffin and cut into sections for staining and subsequent inspection by light microscopy. Current methods of IHC use either direct labeling or secondary antibody-based or hapten-based labeling. Examples of known IHC systems include, for example, EnVision™ (DakoCytomation), Powervision® (Immunovision, Springdale, Ariz.), the NBA™ kit (Zymed Laboratories Inc., South San Francisco, Calif.), HistoFine® (Nichirei Corp, Tokyo, Japan). In some embodiment, a tissue section (e.g. a skin sample) may be mounted on a slide or other support after incubation with antibodies directed against the proteins encoded by the genes of interest. Then, microscopic inspections in the sample mounted on a suitable solid support may be performed. For the production of photomicrographs, sections comprising samples may be mounted on a glass slide or other planar support, to highlight by selective staining the presence of the proteins of interest. Therefore IHC samples may include, for instance: (a) preparations comprising cumulus cells (b) fixed and embedded said cells and (c) detecting the proteins of interest in said cells samples. In some embodiments, an IHC staining procedure may comprise steps such as: cutting and trimming tissue, fixation, dehydration, paraffin infiltration, cutting in thin sections, mounting onto glass slides, baking, deparaffination, rehydration, antigen retrieval, blocking steps, applying primary antibodies, washing, applying secondary antibodies (optionally coupled to a suitable detectable label), washing, counter staining, and microscopic examination.

In some embodiments, the agent capable of restoring the expression of desmoglein-1 is polynucleotide encoding for desmoglein 1. In some embodiments, the polynucleotide comprises a nucleic acid sequence having at least 90% of identity with SEQ ID NO:1.

According to the invention a first nucleic acid sequence having at least 90% of identity with a second nucleic acid sequence means that the first sequence has 90; 91; 92; 93; 94; 95; 96; 97; 98; 99 or 100% of identity with the second amino acid sequence. Sequence identity is frequently measured in terms of percentage identity (or similarity or homology); the higher the percentage, the more similar are the two sequences. Methods of alignment of sequences for comparison are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations. By way of example, the alignment tools ALIGN (Myers and Miller, CABIOS 4:11-17, 1989) or LFASTA (Pearson and Lipman, 1988) may be used to perform sequence comparisons (Internet Program® 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

In some embodiments, the polynucleotide of the present invention is included in a suitable vector, such as a plasmid, cosmid, episome, artificial chromosome, phage or a viral vector. Typically, the vector is a viral vector which is an adeno-associated virus (AAV), a retrovirus, bovine papilloma virus, an adenovirus vector, a lentiviral vector, a vaccinia virus, a polyoma virus, or an infective virus. In some embodiments, the vector is an AAV vector. As used herein, the term “AAV vector” means a vector derived from an adeno-associated virus serotype, including without limitation, AAV1, AAV2, AAV3, AAV4, AAV5, AAV6, AAV7, AAV8, AAV9, and mutated forms thereof AAV vectors can have one or more of the AAV wild-type genes deleted in whole or part, preferably the rep and/or cap genes, but retain functional flanking ITR sequences. Retroviruses may be chosen as gene delivery vectors due to their ability to integrate their genes into the host genome, transferring a large amount of foreign genetic material, infecting a broad spectrum of species and cell types and for being packaged in special cell-lines. In order to construct a retroviral vector, a nucleic acid encoding a gene of interest is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV 1, HIV 2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are known in the art, see, e.g. U.S. Pat. Nos. 6,013,516 and 5,994,136, both of which are incorporated herein by reference. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest. Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene into that packaging cell yields a producer cell which releases infectious viral particles carrying the foreign gene of interest. The env preferably is an amphotropic envelope protein which allows transduction of cells of human and other species. Typically, the polynucleotide or the vector of the present invention include “control sequences”, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell. Another nucleic acid sequence, is a “promoter” sequence, which is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

As used herein the term “inhibitor of NF-κB signaling pathway” refers to any compound that is capable of inhibiting the NF-κB signaling pathway. In some embodiments, the inhibitor of NF-κB signaling pathway is selected from a group consisting of the following compounds: substituted resorcinols, (E)-3-(4-methylphenylsulfonyl)-2-propenenitrile (such as “Bay 11-7082,” commercially available from Sigma-Aldrich of St. Louis, Mo.), tetrahydrocurcuminoids (such as Tetrahydrocurcuminoid CG, available from Sabinsa Corporation of Piscataway, N.J.), and combinations thereof. In some embodiments, the inhibitor of NF-κB signaling pathway is a substituted resorcinol. Resorcinol is a dihydroxy phenol compound (i.e., 1,3 dihydroxybenzene). As used herein, “substituted resorcinol” means resorcinol comprising at least one substituent in the 2, 4, 5, or 6 position. Thus, the substituted resorcinol may have as few as one and as many as four substituents. Particularly suitable substituted resorcinols include 4-hexyl resorcinol and 4-octylresorcinol, particularly 4-hexyl resorcinol. 4-Hexyl resorcinol is commercially available as “SYNOVEA HR” from Sytheon of Lincoln Park, N.J. 4-Octylresorcinol is commercially available from City Chemical LLC of West Haven, Conn. Examples of suitable substituted resorcinols comprising cyclic aliphatic substituents joining the 2 and 3 positions include Zearalanone and β-Zearalanol. An example of a dihalide-substituted resorcinol is 2,6-dichlororesorcinol. An example of a dinitroso-substituted resorcinol is 2,4-dinitrososorcinol. Substituted resorcinols are prepared by means known in the art, for example, using techniques described in U.S. Pat. No. 4,337,370, the contents of which are incorporated herein by reference.

In some embodiments, examples of inhibitors of NF-κB signaling pathway include those described in the international patent application WO2010047127. In some embodiments, the inhibitor of NF-κB signaling pathway is selected from a group consisting of
(12aS,13S)-5,6,7-trimethoxy-9,10,11,12,12a,13-hexahydro-9a-aza-cyclopenta[b]triphenylene-3,13-diol;
(12aR,13R)-5,6,7-trimethoxy-9,10,11,12,12a,13-hexahydro-9a-aza-cyclopenta[b]triphenylene-3,13-diol;
(12aS,13S)-9,10,11,12,12a,13-hexahydro-9a-aza-cyclopenta[b]triphenylene-3,13-diol;
(12aS,13S)-6-fluoro-9,10,11,12,12a,13-hexahydro-9a-aza-cyclopenta[b]triphenylene-3,13-diol;
acetic acid(12aS,13S)-3-hydroxy-6,7-dimethoxy-9,10,11,12,12a,13-hexahydro-9a-aza-cyclopenta[b]triphenylene-13-yl ester;
6,7-dimethoxy-12a-methyl-9,10,11,12,12a,13-hexahydro-9a-aza-cyclopenta[b]triphenylene-3,13-diol;
(S)-13-amino-6,7-dimethoxy-9,10,11,12,12a,13-hexahydro-9a-aza-cyclopenta[b]triphenylene-3-ol;
(12aS,13S)-6,7-methylenedioxy-9,10,11,12,12a,13-hexahydro-9a-aza-cyclopenta[b]triphenylene-3,13-diol;
(12aS,13S)-6,7-isopropylidenedioxy-9,10,11,12,12a,13-hexahydro-9a-aza-cyclopenta[b]triphenylene-3,13-diol;
(12aS,13S)-6,7-diethoxy-9,10,11,12,12a,13-hexahydro-9a-aza-cyclopenta[b]triphenylene-3,13-diol;
(S)-6,7-dimethoxy-9,10,11,12,12a,13-hexahydro-9a-aza-cyclopenta[b]triphenylene-3-ol;
(R)-6,7-dimethoxy-9,10,11,12,12a,13-hexahydro-9a-aza-cyclopenta[b]triphenylene-3-ol;
(S)-6,7-methylenedioxy-9,10,11,12,12a,13-hexahydro-9a-aza-cyclopenta[b]triphenylene-3-ol;
(S)-6,7-diethoxy-9,10,11,12,12a,13-hexahydro-9a-aza-cyclopenta[b]triphenylene-3-ol;
(12aS,13S)-2,3-dimethoxy-9,10,11,12,12a,13-hexahydro-9a-aza-cyclopenta[b]triphenylene-6,13-diol;
(S)-2-chloro-6,7-dimethoxy-9,10,11,12,12a,13-hexahydro-9a-aza-cyclopenta[b]triphenylene-3-ol;
(S)-4-chloro-6,7-dimethoxy-9,10,11,12,12a,13-hexahydro-9a-aza-cyclopenta[b]triphenylene-3-ol;
(S)-2,4-dichloro-6,7-dimethoxy-9,10,11,12,12a,13-hexahydro-9a-aza-cyclopenta[b]triphenylene-3-ol;
(S)-4-fluoro-6,7-dimethoxy-9,10,11,12,12a,13-hexahydro-9a-aza-cyclopenta[b]triphenylene-3-ol;
(S)-2-fluoro-6,7-dimethoxy-9,10,11,12,12a,13-hexahydro-9a-aza-cyclopenta[b]triphenylene-3-ol; and
(S)-6,7-dimethoxy-2,4-dimethyl-9,10,11,12,12a,13-hexahydro-9a-aza-cyclopenta[b]triphenylene-3-ol.

Other examples of inhibitors of NF-κB signaling pathway include, without limitation, α-lipoic acid, α-tocopherol, allicin, 2-amino-1-methyl-6-phenylimidazo[4,5-b]pyridine, anetholdithiolthione, apocynin, 5,6,3′,5′-tetramethoxy 7,4′-hydroxyflavone, astaxanthin, benidipine, bis-eugenol, bruguiera gymnorrhiza compounds, butylated hydroxyanisole, cepharanthine, caffeic acid phenethyl ester, carnosol, β-carotene, carvedilol, catechol derivatives, chlorogenic acid, cocoa polyphenols, curcumin, dehydroepiandrosterone and dehydroepiandrosterone sulfate, dibenzylbutyrolactone lignans, diethyldithiocarbamate, diferoxamine, dihydroisoeugenol, dihydrolipoic acid, dilazep+fenofibric acid, dimethyldithiocarbamates, dimethylsulfoxide, disulfiram, ebselen, edaravone, epc-k1, epigallocatechin-3-gallate, ergothioneine, ethylene glycol tetraacetic acid, flavonoids (Crataegus; boerhaavia diffusa root; xanthohumol), α-glutamylcysteine synthetase, ganoderma lucidum polysaccharides, garcinol, ginkgo biloba extract, hematein, 23-hydroxyursolic acid, iron tetrakis, isovitexin, kangen-karyu extract, I-cysteine, lacidipine, lazaroids, lupeol, magnolol, maltol, manganese superoxide dismutase, extract of the stem bark of mangifera indica I, melatonin, mulberry anthocyanins, n-acetyl-1-cysteine, nacyselyn, nordihydroguaiaritic acid, ochnaflavone, orthophenanthroline, hydroquinone, tert-butyl hydroquinone, phenylarsine oxide, phyllanthus urinaria, pyrrolinedithiocarbamate, quercetin (low concentrations), redox factor 1, rotenone, roxithromycin, s-allyl-cysteine, sauchinone, spironolactone, strawberry extracts, taxifolin, tempol, tepoxaline, vitamin C, vitamin B6, vitamin E derivatives, α-torphryl succinate, α-torphryl acetate, 2,2,5,7,8-pentamethyl-6-hydroxychromane, yakuchinone α and β, n-acetyl-leucinyl-leucynil-norleucynal, n-acetyl-leucinyl-leucynil-methional, carbobenzoxyl-leucinyl-leucynil-norvalinal, carbobenzoxyl-leucinyl-leucynil-leucynal, lactacystine, β-lactone, boronic acid peptide, ubiquitin ligase inhibitors, bortezomib, salinosporamide α, cyclosporin α, tacrolimus, deoxyspergualin, 15 deoxyspergualin, analogs of 15-deoxyspergualin, n-acetyl-dl-phenylalanine-β-naphthylester, n-benzoyl 1-tyrosine-ethylester, 3,4-dichloroisocoumarin, diisopropyl fluorophosphate, n-α-tosyl-1-phenylalanine chloromethyl ketone, n-α-tosyl-1-lysine chloromethyl ketone, desloratadine, salmeterol, fluticasone propionate, protein-bound polysaccharide from basidiomycetes, calagualine, golli bg21, npm-alk oncoprotein, Iy29, ly30, ly294002, evodiamine, rituximab, kinase suppressor of ras, pefabloc, rocaglamides, betaine, tnap, geldanamycin, grape seed proanthocyanidins, pomegranate fruit extract, tetrandine, 4(2′-aminoethyl)amino-1,8-dimethylimidazo(1,2-α) quinoxaline, 2-amino-3-cyano-4-aryl-6-(2-hydroxy-phenyl)pyridine derivatives, acrolein, anandamide, as602868, cobrotoxin, dihydroxyphenylethanol, herbimycin α, inhibitor 22, isorhapontigenin, manumycin α, mlb120, nitric oxide, nitric oxide donating aspirin, thienopyridine, acetyl-boswellic acids, β-carboline, cyl-19s, cyl-26z, synthetic α-methylene-β-butyrolactone derivatives, 2-amino-6-[2-(cyclopropylmethoxy)-6-hydroxyphenyl]-4-piperidin-4-yl nicotinonitrile, plant compound α, flavopiridol, cyclopentones, jesterone dimmer, ps-1 145, 2-[(aminocarbonyl)amino]-5-acetylenyl-3-thiophenecarboxamides, V acetoxychavicol acetate, apigenin, cardamomin, synthetic triterpenoid, chs 828 (anticancer drug), diosgenin, furonaphthoquinone, guggulsterone, heparin-binding epidermal growth factor-like growth factor, falcarindol, hepatocyte growth factor, honokiol, hypoestoxide, γ-mangostin, garcinone β, kahweol, kava derivatives, ml120b, mx781 (retinoid antagonist), n-acetylcysteine, nitrosylcobalamin (vitamin B12 analog), non-steroidal anti-inflammatory drugs (NSAIDs), hepatits c virus ns5b, panl (aka na1p2 or pypaf2), n-(4-hydroxyphenyl) retinamide, sulforaphane, phenylisothiocyanate, survanta, piceatannol, 5-hydroxy-2-methyl-1,4-naphthoquinone, pten (tumor suppressor), theaflavin, tilianin, zerumbone, silibinin, sulfasalazine, sulfasalazine analogs, rosmarinic acid, staurosporine, γ tocotrienol, wedelolactone, betulinic acid, ursolic acid, thalidomide, interleukin-10, mollusum contagiosum virus mc159 protein, monochloramine, glycine chloramine, anethole, anti-thrombin III, artemisia vestita, aspirin, sodium salicylate, azidothymidine, baoganning, e3((4-methylphenyl)-sulfonyl)-2-propenenitrile, e3((4-t-butylphenyl)-sulfonyl)-2-propenenitrile, benzyl isothiocyanate, cyanidin 3-o-glucoside, cyanidin 3-o-(2(g)-xylosylrutinoside, cyanidin 3-o-rutinoside, buddlejasaponin IV, cacospongionolide β, carbon monoxide, carboplatin, cardamonin, chorionic gonadotropin, cycloepoxydon, 1-hydroxy-2-hydroxymethyl-3-pent-1-enylbenzene, decursin, dexanabinol, digitoxin, diterpenes (synthetic), docosahexaenoic acid, extensively oxidized low density lipoprotein, 4-hydroxynonenal, fragile histidine triad protein, gabexate mesilate, [6]-gingerol, casparol, imatanib, glossogyne tenuifolia, ibuprofen, indirubin-3′-oxime, interferon-α, licorice extracts, methotrexate, nafamostat mesilate, oleandrin, omega 3 fatty acids, panduratin α, petrosaspongiolide m, pinosylvin, plagius flosculosus extract polyacetylene spiroketal, phytic acid, prostaglandin α1, 20(s)-protopanaxatriol, rengyolone, rottlerin, saikosaponin-d, saline (low Na+ istonic), salvia miltiorrhizae water-soluble extract, pseudochelerythrine, 13-methyl-[1,3]-benzodioxolo-[5,6-c]-1,3-dioxolo-4,5 phenanthridinium), scoparone, silymarin, socsi, statins, sulindac, thi 52 (1-naphthylethyl-6,7-dihydroxy-1,2,3,4-tetrahydroisoquinoline), 1,2,4-thiadiazolidine derivatives, vesnarinone, xanthoangelol d, yc-1, yopj, acetaminophen, activated protein c, alachlor, α-melanocyte-stimulating hormone, amentoflavone, artemisia capillaris thunb extract, artemisia iwayomogi extract, 1-ascorbic acid, antrodia camphorate, aucubin, baicalein, β-lapachone, blackberry extract, buchang-tang, capsaicin, catalposide, core protein of hepatitis c virus, cyclolinteinone, diamide, dihydroarteanniun, dobutamine, e-73 (cycloheximide analog), ecabet sodium, emodin, ephedrae herba, equol, erbstatin, estrogen, ethacrynic acid, fosfomycin, fungal gliotoxin, gamisanghyulyunbueum, genistein, genipin, glabridin, glimepiride, glucosamine sulfate, glutamine, gumiganghwaltang, heat shock protein-70, hypochlorite, interleukin-13, isomallotochromanol, isomallotochromene, vaccinia virus protein, kochia scoparia fruit, leflunomide metabolite, losartin, 5′-methylthioadenosine, momordin I, morinda officinalis extract, murri gene product, neurofibromatosis-2 protein, uO126, penetratin, pervanadate, β-phenylethyl and 8-methylsulphinyloctyl isothiocyanates, phenytoin, platycodin saponins, polymyxin β, poncirus trifoliata fruit extract, probiotics, pituitary adenylate cyclase-activating polypeptide, prostaglandin 15-deoxy-delta(12,14)-pgj(2), resiniferatoxin, sabaeksan, saccharomyces boulardii anti-inflammatory factor, sesquiterpene lactones (parthenolide; ergolide; guaianolides), st2 (interleukin-1-like receptor secreted form), thiopental, tipifarnib, titanium, tnp-470, stinging nettle (urtica dioica) plant extracts, trichomomas vaginalis infection, triglyceride-rich lipoproteins, ursodeoxycholic acid, xanthium strumarium I, vasoactive intestinal peptide, HIV-1 vpu protein, epoxyquinone α monomer, ro106-9920, conophylline, mol 294, perrilyl alcohol, mast205, rhein, 15-deoxy-prostaglandin j(2), antrodia camphorata extract, β-amyloid protein, surfactant protein α, dq 65-79 (aa 65-79 of the α helix of the alpha-chain of the class Il HLA molecule dqa03011), c5a, glucocorticoids (dexamethasone, prednisone, methylprednisolone), interleukin-10, interleukin-1 1, α-pinene, vitamin D, foxij, dioxin, agastache rugosa leaf extract, alginic acid, astragaloside iv, atorvastatin, blue honeysuckle extract, n(1)-benzyl-4-methylbenzene-1,2-diamine, buthus martensi karsch extract, canine distemper virus protein, carbaryl, celastrol, chiisanoside, dehydroxymethylepoxyquinomicin, dipyridamole, diltiazem, eriocalyxin β, estrogen enhanced transcript, gangliosides, glucorticoid-induced leucine zipper protein, harpagophytum procumbens extracts, heat shock protein 72, hirsutenone, indole-3-carbinol, jm34 (benzamide derivative), 6-hydroxy-7-methoxychroman-2-carboxylic acid phenylamide, leptomycin β, levamisole, 2-(4-morpholynl) ethyl butyrate hydrochloride, nls cell permeable peptides, 2′,8″-biapigenin, nucling, o,o′-bismyristoyl thiamine disulfide, oregonin, 1,2,3,4,6-penta-o-galloyl-β-d-glucose, platycodi radix extract, phallacidin, piperine, pitavastatin, pn-50, rela peptides (p1 and p6), retinoic acid receptor-related orphan receptor-α, rhubarb aqueous extract, rolipram, salvia miltiorrhoza bunge extract, sc236 (a selective cox-2 inhibitor), selenomethionine, sophorae radix extract, sopoongsan, sphondin, younggaechulgam-tang, zud protein, zas3 protein, clarithromycin, fluvastatin, leflunomide, oxidized 1-palmitoyl-2-arachidonoyl-sn-glycero-3-phosphorylcholine, serratamolide, moxifloxacin, sorbus commixta cortex, cantharidin, cornus officinalis extract, neomycin, omapatrilat, enalapril, cgs 25462, onconase, paeoniflorin, rapamycin, sargassum hemiphyllum methanol extract, shenfu, tripterygium polyglycosides, triflusal, hepatoma protein, andrographolide, melittin, 1′-acetoxychavicol acetate, 2-acetylaminofluorene, actinodaphine, adiponectin, nicotinamide, 3-aminobenzamide, 7-amino-4-methylcoumarin, amrinone, angiopoietin-1, anthocyanins, sequiterpene lactones, artemisinin, atrial natriuretic peptide, atrovastat, avra protein, baicalein, benfotiamine, β-catenin, biliverdin, bisphenol α, bovine serum albumin, brazilian, bromelain, calcium/calmodulin-dependent kinase kinase, calcitriol, campthothecin, sutherlandia frutescens, caprofin, capsiate, carbocisteine, cat's claw bark, maca, celecoxib, germcitabine, cheongyeolsaseuptang, chitosan, ciclosporin, cinnamaldehyde, 2-methoxycinnamaldehyde, 2-hydroxycinnamaldehyde, guaianolide 8-deoxylactucin, chlorophyllin, chondrotin sulfate proteoglycan degradation product, clarithromycin, cloricromene, commerical peritoneal dialysis solution, compound K, 6-hydroxy-7-methoxychroman̂-carboxylic acid phenylamide, cryptotanshinone, cyanoguanidine, cytochalasin d, da-9201 (from black rice), danshenshu, decoy oligonucleotides, diarylheptanoid 7-(4′-hydroxy-3′-methoxyphenyl)-1-phenylhept-4-en-3-one, α-difluoromethylornithine, dim/13c, diterpenoids from isodon rubescens or liverwort jungermannia, 4,10-dichloropyrido[5,6:4,5]thieno[3,2-d′:3,2-d]-1,2,3-ditriazine, e3330, ent-kaurane diterpenoids, epinastine hydrochloride, epoxyquinol α, erythromycin, evans blue, fenoldopam, fexofenadine hydrochloride, fibrates, fk778, flunixin meglumine, flurbiprofen, fomes fomentarius methanol extracts, fucoidan, glycoprotein-120, gallic acid, ganoderma lucidum, homeobox protein, geranylgeraniol, ghrelin, ginkgolide β, glycyrrhizin, halofuginone, helenalin, herbal compound 861, HIV-1 resistance factor, hydroxyethyl starch, hydroxyethylpuerarin, hypercapnic acidosis, hypericin, interleukin 4, 1κB-like proteins, imd-0354, insulin-like growth factor binding protein-3, jsh-21 (n1-benzyl-4-methylbenzene-1,2-diamine), kamebakaurin, kaposi's sarcoma-associated herpesvirus k1 protein, ketamine, kt-90 (morphine synthetic derivative), linoleic acid, lithospermi radix, lovastatin, macrolide antibiotics, mercaptopyrazine, 2-methoxyestradiol, 6 (methylsulfinyl)hexyl isothiocyanate, metals (chromium, cadmium, gold, lead, mercury, zinc, arsenic), mevinolin, monomethylfumarate, moxifloxacin, myricetin, myxoma virus mnf, ndppi, n-ethyl-maleimide, naringen, nicorandil, nicotine, nilvadipine, nitrosoglutathione, extracts of ochna macrocalyx bark, leucine-rich effector proteins of salmonella & shigella, omega-3 fatty acids oridonin 1,2,3,4,6-penta-o-galloyl-beta-d-glucose, interferon inducible protein, p21 (recombinant), peptide nucleic acid-DNA decoys, pentoxifylline (1-(5′-oxohexyl) 3,7-dimetylxanthine, peptide yy, pepluanone, perindopril, 6(5h)-phenanthridinone and benzamide, phenyl-n-tert-butylnitrone, phyllanthus amarus extracts, protein inhibitor of activatated stati, pioglitazone, pirfenidone, polyozellin, prenylbisabolane 3, proopiomelanocortin, prostaglandin e2, protein-bound polysaccharide, pypafl protein, pyridine n-oxide derivatives, pyrithione, quinadril, quinic acid, raf kinase inhibitor protein, rapamycin, raloxifene, raxofelast, rebamipide, rhus verniciflua stokes fruits 36 kda glycoprotein, ribavirin, rifamides, ritonavir, rosiglitazone, sanggenon c, santonin diacetoxy acetal derivative, secretory leucoprotease inhibitor, n-(p-coumaroyl) serotonin, sesamin, simvastatin, sinomenine, sirti deacetylase overexpression, siva-1, sm-7368, solana nigrum I, 150 kda glycoprotein, sun c8079, tanacetum larvatum extract, tansinones, taurine+niacine, thiazolidinedione mcc-555, trichostatin α, triclosan plus cetylpyridinium chloride, triptolide, tyrphostin ag-126, uteroglobin, vascular endothelial growth factor, verapamil, withaferin α, 5,7-dihydroxy-8-methoxyflavone, xylitol, yan-gan-wan, yin-chen-hao, yucca schidigera extract, amp-activated protein kinase, apc0576, artemisia sylvatica, bsasm, bifodobacteria, bupleurum fruticosum phenylpropanoids, ebv protein, chromene derivatives, dehydroevodiamine, 4′-demethyl-6-methoxypodophyllotoxin, ethyl 2-[(3-methyl-2,5-dioxo(3-pyrrolinyl))amino]-4-(trifluoromethyl) pyrimidine-5-carboxylate, cycloprodigiosin hycrochloride, dimethylfumarate, fructus benincasae recens extract, glucocorticoids (dexametasone, prednisone, methylprednisolone), gypenoside xlix, histidine, HIV-1 protease inhibitors (nelfinavir, ritonavir, or saquinavir), 4-methyl-N1-(3-phenyl-propyl)-benzene-1,2-diamine, kwei ling ko, ligusticum chuanxiong hort root, nobiletin, NFKB repression factors, phenethylisothiocyanate, 4-phenylcoumarins, phomol, pias3, pranlukast, psychosine, quinazolines, resveratrol, ro31-8220, saucerneol d and saucerneol e, sb203580, tranilast, 3,4,5-trimethoxy-4′-fluorochalcone, uncaria tomentosum plant extract, mesalamine, mesuol, pertussis toxin binding protein, 9-aminoacridine derivatives (including the antimalaria drug quinacrine), adenosine and cyclic amp, 17-allylamino-17-demethoxygeldanamycin, 6-aminoquinazoline derivatives, luteolin, manassantins α and β, paramyxovirus sh gene products, qingkailing, shuanghuanglian, smilax bockii warb extract, tetracyclic a, tetrathiomolybdate, trilinolein, troglitazone, witheringia solanacea leaf extracts, wortmannin, α-zearalenol, antithrombin, rifampicin, and mangiferin

As used herein, the term “IL-6”, “IL-8”, “IL-1beta” and “TSLP” have their general meaning in the art and refers to interleukin-6, interleukin-8, interleukin 1beta, thymic stromal lymphopoietin and respectively.

In some embodiments, the inhibitor of IL-6, IL-8, IL-1beta or TSLP is an antibody. As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/11161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388. In some embodiments, the antibody is a humanized antibody. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. In some embodiments, the antibody is a fully human antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans. In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of IL-6, IL-8, IL-1beta or TSLP mRNA by binding thereto and thus preventing protein translation or increasing mRNA degradation, thus decreasing the level of IL-6, IL-8, IL-1beta or TSLP, and thus activity, in a cell. For example, antisense oligonucleotides of at least about 15 bases and complementary to unique regions of the mRNA transcript sequence encoding IL-6, IL-8, IL-1beta or TSLP can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. IL-6, IL-8, IL-1beta or TSLP gene expression can be reduced by contacting a subject or cell with a small double stranded RNA (dsRNA), or a vector or construct causing the production of a small double stranded RNA, such that IL-6, IL-8, IL-1beta or TSLP gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing IL-6, IL-8, IL-1beta or TSLP. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

By a “therapeutically effective amount” of the active agent as above described is meant a sufficient amount to provide a therapeutic effect. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

According to the invention, the active agent is administered to the subject in the form of a pharmaceutical composition. Typically, the active agent may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The active agent can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### EXAMPLE

Methods:

Case Reports

Patient#1, a 13-year-old boy, was referred for life-long desquamative erythroderma. He was born to healthy non-consanguineous parents. Since birth, he had presented with sparse scalp and body hair, without abnormal hair shaft under the light microscope. He also had dysplastic enamel, numerous caries, dystrophic nails, and reduced sweating. At the age of one year, he developed painful palmoplantar keratoderma (PPK). Erythrodermic features were combined with recurrent, painful, erythematous skin flares often triggered by infections. Episodes of aseptic pustular psoriasiform dermatitis, nail and hair loss and regrowth were noted. He displayed failure to thrive associated to eosinophilic esophagitis and colitis, and a variety of food allergies [with an elevated total serum IgE level of 2968 kIU/mL (N<114)]. Neither primary nor secondary immunodeficiencies could be detected. The patient also experienced episodes of spontaneously remitting cytolytic hepatitis and an unexplained episode of spontaneously resolving acute pancreatitis at the age of 13 years. Cardiac monitoring revealed an asymptomatic, biventricular, dilated cardiomyopathy. Cardiac MRI showed fibrosis of the laterobasal segment of the left ventricle, with right ventricular dilatation in favor of a biventricular arrhythmogenic cardiomyopathy. Histopathological examination of the skin revealed epidermal acanthosis and extensive acantholysis, in the lower part of the epidermis and a lymphocytic infiltration of the dermis. Upon ultrastructural examination multiple abnormal clusters of desmosomes in the upper epidermis and a reduced number of desmosomes in the lower epidermis were observed. Although keratin filaments were normally attached to the desmosomes, the inner plaque was missing.

Patient#2, a 9-year-old boy, born to non-consanguineous healthy parents, presented with permanent desquamative erythroderma developed at 18 months. His hair had been woolly and sparse since birth. At the age of 2 years, he developed diffuse PPK, dystrophic toenails and dysplastic enamel with absence of definitive teeth. He presented with a combination of painful and erythrodermic flares and episodes of aseptic pustular psoriasiform dermatitis. Compared with Patient#1, his skin was less red and less thickened. There was no clinical history of allergy and the total serum IgE level was mildly increased. At the age of 6 years, sudden cardiac arrest revealed left dominant arrhythmogenic cardiomyopathy. Due to severe heart failure at 9 years, he underwent cardiac transplantation. Histopathological examination of the explanted heart showed the characteristic fibro-fatty myocardial infiltration described in arrhythmogenic dysplasias with no significant inflammation.

Molecular Genetics Analysis

DNA was extracted from peripheral blood lymphocytes using the Nucleon BACC3 DNA extraction kit (GE Healthcare, Piscataway, N.J., USA), according to the manufacturer's instructions. Genomic DNA (1 μg) samples from Patient#1 and his parents underwent whole exome sequencing. The exons were captured with an in-solution enrichment methodology (SureSelect Human All Exon Kits Version 3, Agilent, Massy, France) using the company's biotinylated oligonucleotide probe library (Human All Exon v3 50 Mb, Agilent). Each genomic DNA fragment was sequenced on a sequencer using the paired-end strategy and an average read length of 75 bases (Illumina HISEQ, Illumina, San Diego, Calif., USA). Image analysis and base calling were performed with Real Time Analysis (RTA) Pipeline version 1.9 with default parameters (Illumina). Sequences were aligned to the human genome reference sequence (hg19 assembly), and SNPs were called based on allele calls and read depth using the CASAVA pipeline (Consensus Assessment of Sequence and Variation 1.8, Illumina). Genetic variations were annotated using an in-house pipeline (IntegraGen), and the results for each sample were made available online to enable their analysis with ERIS Integragen Software (http://eris.integragen.com/). The DSP variant p.H586P was confirmed by Sanger sequencing with specific primers for exon 14 of the DSP gene. For Patient#2 and his relatives, the 24 exons of DSP were amplified by PCR with specific primers. PCR products were sequenced using the Sanger method with the BigDye™ Terminator Cycle Sequencing Ready Reaction Kit (version 3.1, Applied Biosystems, Foster City, Calif., USA) and analyzed with SeqScape® Analysis software (version 3.0, Applied Biosystems).

RNA Extraction, RT-PCR and Real-Time PCR

Total RNAs were isolated from cultured keratinocytes, HEK293T cells and frozen skin biopsies from the two patients and controls using the RNeasy Plus Minikit (Qiagen GmbH, Hilden, Germany), according to the manufacturer's instructions. RNA samples were reverse-transcribed into cDNA using the High Capacity cDNA Reverse Transcriptase Kit (Applied Biosystems, Foster City, Calif., USA). Real-time PCR was carried out using the Fast SYBR Green PCR Master Mix (PE Applied Biosystems) on an ABI prism 7000 (PE Applied Biosystems). RT-qPCR primers were designed using the sequences available in Ensembl and spanned an intron-exon boundary. The amounts of the various mRNAs were normalized against the amount of beta actin RNA measured by RT-qPCR in each sample. The results were analyzed with DataAssist® (version 3.01, Applied Biosystems), which uses the comparative Ct (ddCt) method.

Inhibition of IKK-2 by ML120B

Keratinocytes from a healthy control and from Patient#1 were seeded into 12-well plates (100 000 cells/well). 24 hours later, keratinocytes were preincubated with ML120B 20 μM at 37° C. for 1 h and then stimulated with 10 ng/ml IL-1β. 24 hours after the stimulation, the cells were pelleted for RNA extraction. ML120B was sent as a gift by Emmanuel Laplantine (Institut Pasteur, Paris, France).

Luciferase NF-κB Reporter Assays

For the NF-κB reporter assay, the HEK293T cells were seeded into 24-well plates. Cells were transfected in triplicate using jetPRIM™ reagent (Polyplus Transfection Inc., New York, N.Y., USA) with increasing doses (100-1000 ng) of DSG1 plasmid [mCherry-Desmoglein1-C-18 was a gift from Michael Davidson (Addgeneplasmid #55029)] or with 250 ng of DSP plasmid [1136-Desmoplakin-GFP was a gift from Kathleen Green (Addgene plasmid #32227)] together with 0.2 μg of a plasmid carrying the firefly luciferase gene under the control of the NF-κB promoter (Igκluc, a gift from Gilles Courtois, Grenoble, France). 16 hours after transfection, cells were stimulated with 10 ng/ml IL-1β or 20 ng/ml TNFα. 8 hours after stimulation, luciferase activity was determined using a dual luciferase assay kit (Promega, Madison, Wis., USA).

Immunoblotting Analysis

HEK293T cells were transiently transfected with increasing doses (100-1000 ng) of DSG1 plasmid (plasmid #55029, Addgene) or with 250 ng of DSP plasmid (plasmid #32227, Addgene), together with 0.2 μg of Igκluc (see above in the “Luciferase NF-κB reporter assays” section), and then lysed in RIPA buffer (150 mMNaCl, 1% NP-40, 0.5% sodium deoxycholate, 0.1% SDS, 50 mMTris-HCl pH 8) with protease inhibitors (Roche Diagnostics GmbH, Mannheim, Germany). Western blotting was performed using mouse anti-DSP I/II antibody (diluted 1:1000, sc-390975, Santa Cruz Biotechnology, Heidelberg, Germany) and rabbit anti-DSG1 antibody (diluted 1:1000, sc-20114, Santa Cruz Biotechnology). Bound antibodies were visualized with horseradish-peroxidase-conjugated antibodies against rabbit or mouse IgG (Santa Cruz Biotechnology) and an Enhanced Chemiluminescence kit (SuperSignal West Dura Extended Duration Substrate, Thermo Scientific, Rockford, Ill., USA).

Lentiviral Transduction

Keratinocytes from a healthy control were seeded into 12-well plates. 12 hours later, keratinocytes were infected with lentivirus containing (or not) DSG1 shRNA (7 μL/well, sc-35224-V Santa Cruz Biotechnology). 24 hours after infection, keratinocytes were stimulated with 10 ng/ml of IL-1β (R&D Systems, Minneapolis Minn., USA). 24 hours after stimulation, the cells were pelleted for RNA extraction. The down-expression of DSG1 was confirmed by RT-PCR.

Retroviral Vector Production

pRetro-DSG1 was sent as a gift by Kathleen Green (Northwestern University, Chicago, Ill., USA). For virus preparation, pRetro-DSG1 or blank vector were co-transfected using jetPRIME™ reagent (Polyplus Transfection Inc.) and packaging vectors pGag/Pol and pVSVG into HEK293T cells. Infectious retroviruses were harvested at 24, 48 and 72 hours post-transfection and filtered through 0.8-μm-pore cellulose acetate filters. Recombinant retroviruses were concentrated by ultracentrifugation (2 hours at 20,000×g) and resuspended in Hank's Balanced Salt Solution. The virus aliquots were frozen and stored at −80° C.

Retroviral Transduction

Keratinocytes from Patient#1 and a healthy control were seeded into 12-well plates (80 000 cells/well). 12 hours later, keratinocytes (20% confluent) were infected with retrovirus containing (or not) the DSG1 construct. 24 hours after infection, keratinocytes were stimulated with 10 ng/ml of IL-1β (R&D Systems). 24 hours after stimulation, the cells were pelleted for RNA extraction. DSG1 expression was confirmed using RT-qPCR.

Immunohistochemistry of Skin and Esophagus Biopsy Specimens

Immunohistochemical reactions were performed on 4-μm-thick frozen tissue sections using rabbit anti-DSG1 antibody (diluted 1:50, sc-20114, Santa Cruz Biotechnology) and mouse anti-DSP I/II antibody (diluted 1:50, sc-390975, Santa Cruz Biotechnology). The secondary antibodies were anti-rabbit Alexa Fluor 546 and anti-mouse Alexa Fluor 488 (Life Technologies, Grand Island, N.Y.), diluted 1:500 in 1% normal goat serum for 1 hour at 37° C. Sections were washed with PBS 1×. Coverslips were mounted with mounting medium with DAPI (Duolink, Olink Biosciences, Uppsala, Sweden). Images were acquired and processed with an LSM700 microscope (Zeiss, Jena, Germany) and Zen Software (Zeiss, Jena, Germany).

Light Microscopy

Skin and heart biopsy specimens were fixed in 10% formalin, embedded in paraffin and processed using standard procedures. Three-μm-thick sections were stained with H&E reagent and examined under the LEICA DFC280 light microscopy (Leica, Buffalo Grove, Ill., USA) at different magnifications. Images were acquired with Leica Application Suite Software.

Electron Microscopy

The skin biopsy sample was immersed in 2.5% glutaraldehyde fixative in 0.1M cacodylate buffer at pH 7.4 for 3 to 5 hours at 4° C., washed thoroughly in cacodylate buffer overnight at 4° C. and then postfixed in 1% osmium tetroxide for 1 hour at room temperature. The skin biopsy slices were then dehydrated in graded ethanol and impregnated with epoxy resin. After selection of suitable areas, the semithin sections were stained with 1% toluidine blue and examined under the light microscope. Ultrathin sections were prepared and stained with uranyl acetate and lead citrate for electron microscopy (Tecnai T12, FEI, Hillsboro, O, USA).

Statistical Analysis

Results were expressed as the mean±standard deviation. Statistical significance was determined using unpaired, two-sample t-tests (equal variance). All data were normally distributed, and the variance was similar in groups that were compared in statistical tests. The threshold for statistical significance was set to p<0.05.

Results

Molecular Genetics

Whole-exome sequencing of DNA extracted from Patient#1 leucocytes revealed the heterozygous de novo missense mutation in exon 14 of the DSP gene, previously observed in the patient described in McAleer et al (c.A1757C/p.H586P).⁶ Clinical similarities between the two patients prompted us to sequence the DSP gene in Patient#2; a distinct heterozygous de novo missense mutation (c.T1828C/p.S610P) was identified.

The two mutations identified were not referenced as polymorphisms in Ensembl, ExAC and Imagine Institute's databases. Both mutated amino acids (H586 and S610) are located in DSP's plakin domain [containing a series of spectrin-like repeats (SRs), each of which is composed of a three-alpha-helix bundle].^{9,10} The affected amino acids have been conserved throughout evolution and are located at the surfaces of alpha helices within SR6. Substitution of H586 or S610 by a proline is expected to induce a kink in the helix and thus perturb DSP's three-dimensional structure.

Altered Expression of Desmosome Components

Immunohistochemical analysis of skin biopsies from patients #1 and #2 showed reductions of DSP and DSG1 expression in the epidermis compared both to a healthy control and to a patient from our department carrying bi-allelic DSP mutations with no skin inflammation. Abnormal cytoplasmic accumulation of DSP and DSG1 proteins was observed in patient keratinocytes. Similarly, immunohistochemical analysis of esophageal samples from Patient#1 revealed low DSP expression and absence of DSG1 expression. In Patient#1, the difference in DSG1 staining detected in the esophagus and the epidermis could be accounted for by tissue-specific expression. The expression of DSP protein was also reduced in the heart of Patient#2. DSG1 is not expressed in heart. Accordingly, DSP and DSG1 protein levels were reduced in the keratinocytes from Patient#1.

In the skin of both patients #1 and #2, the amount of DSP mRNA was reduced by 84% and 58%, respectively, compared to controls. The level of DSG1 mRNA in epidermis of patients #1 and #2 was 88% and 60% lower than in control respectively. The level of mRNA of the main desmosome proteins, such as PG and PKP1, was also reduced.

Enhanced NF-κB-Mediated Inflammation in Patient Keratinocytes

Abnormally high levels of mRNAs encoding pro-inflammatory cytokines [IL6 (20-fold), IL8 (3-fold), and IL-1β (2.5-fold)], three NF-κB target genes, and the pro-allergic TH2 cytokine TSLP (1.8-fold) were found in the Patient#1 keratinocytes. Overexpression of IL6 was confirmed by ELISA. In contrast, mRNA levels of TNFα and other pro-TH2 cytokines (IL13 and CCL5, data not shown) were not elevated. Lastly, IL4 and IL5 transcripts were not detected in keratinocytes for either Patient#1 or the controls. Inhibition of the NF-κB signaling pathway, via ML120B which selectively targets the catalytic subunit of the IKK complex, IKK-2, restored the normal production of IL8 by Patient#1 keratinocytes.

DSG1 Inhibits NF-κB-Mediated Inflammation

Considering the primitive DSG1 deficiency reported in SAM syndrome (see the discussion below) and its drastically reduced expression in our patients, we hypothesized that DSG1 could play a role in the inflammatory phenotype. We found that DSG1 led to an inhibition of NF-κB reporter activity, in a dose-dependent manner, following stimulation by IL-1β or TNFα in HEK293T cells This inhibition was correlated with the downregulation of IL6 and IL8 upon transfection of the DSG1-encoding plasmid. No inhibition was observed following transfection of a DSP-encoding plasmid.

Then, we infected control keratinocytes with a lentivirus containing an shRNA against DSG1, which induce a partial silencing of DSG1 (32%, on average). This partial silencing of DSG1 enhanced transcription of the genes coding for IL6, IL8, IL-1β, TNFα and TSLP in unstimulated keratinocytes or following stimulation by IL-1β. Finally, in an attempt to rescue the cellular phenotype, we introduced WT-DSG1, by retroviral transduction, into Patient#1 keratinocytes. Genetic complementation restored IL8 production in Patient#1 keratinocytes compared to control.

Discussion:

Here, we report on two unrelated patients with severe dermatitis and loss of epithelial barrier integrity related to two different, heterozygous mutations in the DSP gene. Both patients presented with a phenotype consisting of SAM syndrome, associated to Ectodermal dysplasia features and arrhythmogenic Cardiomyopathy. For this reason, SAMEC appears the most appropriate term for the characterization of the patients. We show that heterozygous mutations in exon 14 of the DSP gene decreased DSG1 expression, which in turn increased NF-κB-mediated inflammation. We demonstrate, for the first time, the pivotal role of DSG1 protein as an inhibitor of skin inflammation via the NF-κB signaling pathway. The deficiency of NF-κB inhibition resulted in a constitutive overexpression of pro-inflammatory cytokines in Patient#1 keratinocytes. Suppression of DSG1 expression in control keratinocytes reproduced the inflammatory phenotype observed in Patient#1's keratinocytes while DSG1 complementation rescued this phenotype.

Supporting the key role of the DSG1 protein in the inflammatory process, inflammation was only observed in tissues and organs where both DSP and DSG1 are concomitantly expressed i.e. epidermis (skin inflammation), liver (hepatitis), pancreas (pancreatitis) and esophagus (eosinophilic esophagitis).^{11,12} On the other hand, no significant inflammation was observed in heart, which expresses DSP but not DSG1. Moreover, normal DSG1 expression was observed in our control patient carrying bi-allelic DSP mutations with absence of skin inflammation.

We also show that DSP mutations disorganized the desmosomal scaffolding. Impaired epithelial barrier is reported in many inflammatory diseases.^{13,14} Therefore, the loss of epidermal barrier integrity might amplify the inflammatory phenotype in our patients.

In addition to our two patients and SAM syndrome, DSG1 deficiency is reported in atopic dermatitis (AD) and Netherton syndrome (NS, MIM#256500).¹⁵⁻¹⁸ Interestingly, AD, NS, SAM syndrome and our patients display chronic inflammatory dermatitis and allergic manifestations. In further support of this role for DSG1, Guerra et al. reported two NS siblings displaying an absence of skin inflammation with a normal DSG1 epidermal staining.¹⁹ Moreover, it has been suggested that impairment of the mucosal barrier and the inflammation observed in eosinophilic esophagitis could be related to DSG1 deficiency.^{20,21} Together our findings and the published data strongly support the pivotal role of DSG1 deficiency in epithelial inflammation.

Beside its structural role, DSG1 protein is involved in epidermal differentiation through several signaling pathways, such as the Erbin/SHOC2/Ras pathway in which Erbin interacts directly with the intracellular domain of DSG1.^{22,23} Erbin inhibits the NF-κB signaling pathway mediated by NOD2, a pattern recognition receptor involved in innate immunity.^{24,25} Therefore, Erbin might conceivably be one of the links between DSG1 and the NF-κB signaling pathway.

Finally, we propose the acronym SAMEC rather than SAM, to reflect the complex, yet related phenotype of our patients: SAM, Ectodermal dysplasia, and arrhythmogenic Cardiomyopathy. The combination of hair, nails, and tooth anomalies supports assignment of SAMEC syndrome to the ectodermal dysplasias group. Prior to the study by McAleer et al, skin inflammation had never been reported in association to DSP mutations.⁶ The skin features of the previously reported patients consisted in isolated PPK or the combination of PPK and hair anomalies, and/or skin fragility. Arrhythmogenic cardiomyopathy has been consistently observed in patients carrying a single DSP mutation in exon 14.⁷ The young age (6 years) of the patient described in McAleer et al at the time of publication might explain his normal cardiac phenotype. More recently, three additional patients carrying a heterozygous DSP mutation in exon 14 were reported to have an erythrokeratodermia-cardiomyopathy syndrome.²⁶ Based on our findings, these four patients are likely to suffer from SAMEC syndrome.^{6,26} Therefore, cardiac monitoring is required in patients presenting with SAM syndrome until the role of DSP mutations has been excluded.

In conclusion, we show here that DSP mutations induce loss of skin barrier function by direct desmosomal disorganization, and deregulation of the inflammation process through a DSG1 deficiency. The pathophysiological mechanism of SAMEC syndrome highlights, for the first time, the direct pivotal role of DSG1 protein as an inhibitor of skin inflammation (and probably other epithelia) via the NF-κB signaling pathway. The deficiency of an epithelial barrier protein, here DSG1, appears to be a crucial link between loss of epithelial barrier integrity and immune dysregulation. Future research will explore the close links between DSG1 and the NF-κB signaling pathway. Targeting the DSG1 protein may open up opportunities for treating SAMEC syndrome and other inflammatory skin diseases associated with DSG1 deficiency.

### REFERENCES

Throughout this application, various references describe the state of the art to which this invention pertains. The disclosures of these references are hereby incorporated by reference into the present disclosure.

1. Palmer C N A, Irvine A D, Terron-Kwiatkowski A, Zhao Y, Liao H, Lee S P, et al. Common loss-of-function variants of the epidermal barrier protein filaggrin are a major predisposing factor for atopic dermatitis. Nat Genet 2006; 38:441-6.

2. Cork M J, Danby S G, Vasilopoulos Y, Hadgraft J, Lane M E, Moustafa M, et al. Epidermal Barrier Dysfunction in Atopic Dermatitis. J Invest Dermatol 2009; 129:1892-908.

3. Samuelov L, Sarig O, Harmon R M, Rapaport D, Ishida-Yamamoto A, Isakov O, et al. Desmoglein 1 deficiency results in severe dermatitis, multiple allergies and metabolic wasting. Nat Genet 2013; 45:1244-8.

4. Has C, Jakob T, He Y, Kiritsi D, Hausser I, Bruckner-Tuderman L. Loss of desmoglein 1 associated with palmoplantar keratoderma, dermatitis and multiple allergies. Br J Dermatol 2015; 172:257-61.

5. Schlipf N A, Vahlquist A, Teigen N, Virtanen M, Dragomir A, Fismen S, et al. Whole exome sequencing identifies novel autosomal recessive DSG1 mutations associated with mild SAM syndrome. Br J Dermatol 2015.

6. McAleer M A, Pohler E, Smith F J D, Wilson N J, Cole C, MacGowan S, et al. Severe dermatitis, multiple allergies, and metabolic wasting syndrome caused by a novel mutation in the N-terminal plakin domain of desmoplakin. J Allergy Clin Immunol 2015; 136:1268-76.

7. Polivka L, Bodemer C, Hadj-Rabia S. Combination of palmoplantar keratoderma and hair shaft anomalies, the warning signal of severe arrhythmogenic cardiomyopathy: a systematic review on genetic desmosomal diseases. J Med Genet 2015.

8. Pasparakis M. Regulation of tissue homeostasis by NF-kappaB signalling: implications for inflammatory diseases. Nat Rev Immuno12009; 9:778-88.

9. Choi H-J, Weis W I. Crystal structure of a rigid four-spectrin-repeat fragment of the human desmoplakin plakin domain. J Mol Biol 2011; 409:800-12.

10. Al-Jassar C, Knowles T, Jeeves M, Kami K, Behr E, Bikker H, et al. The nonlinear structure of the desmoplakin plakin domain and the effects of cardiomyopathy-linked mutations. J Mol Biol 2011; 411:1049-61.

11. Cao Y, Chang H, Li L, Cheng R-C, Fan X-N. Alteration of adhesion molecule expression and cellular polarity in hepatocellular carcinoma. Histopathology 2007; 51:528-38.

12. Ramani V C, Hennings L, Haun R S. Desmoglein 2 is a substrate of kallikrein 7 in pancreatic cancer. BMC Cancer 2008; 8:373.

13. Kobielak A, Boddupally K. Junctions and inflammation in the skin. Cell Commun Adhes 2014; 21:141-7.

14. Irvine A D, McLean W H I. Breaking the (un)sound barrier: filaggrin is a major gene for atopic dermatitis. J Invest Dermatol 2006; 126:1200-2.

15. Leung D Y M. New insights into atopic dermatitis: role of skin barrier and immune dysregulation. Allergol Int Off J Jpn Soc Allergol 2013; 62:151-61.

16. Broccardo C J, Mahaffey S, Schwarz J, Wruck L, David G, Schlievert P M, et al. Comparative proteomic profiling of patients with atopic dermatitis based on history of eczema herpeticum infection and Staphylococcus aureus colonization. J Allergy Clin Immunol 2011; 127:186-93, 193.e1-11.

17. Chavanas S, Bodemer C, Rochat A, Hamel-Teillac D, Ali M, Irvine A D, et al. Mutations in SPINK5, encoding a serine protease inhibitor, cause Netherton syndrome. Nat Genet 2000; 25:141-2.

18. Fortugno P, Furio L, Teson M, Berretti M, El Hachem M, Zambruno G, et al. The 420K LEKTI variant alters LEKTI proteolytic activation and results in protease deregulation: implications for atopic dermatitis. Hum Mol Genet 2012; 21:4187-200.

19. Guerra L, Fortugno P, Pedicelli C, Mazzanti C, Proto V, Zambruno G, et al. Ichthyosis Linearis Circumflexa as the Only Clinical Manifestation of Netherton Syndrome. Acta Derm Venereol 2015; 95:720-4.

20. Sherrill J D, Kc K, Wu D, Djukic Z, Caldwell J M, Stucke E M, et al. Desmoglein-1 regulates esophageal epithelial barrier function and immune responses in eosinophilic esophagitis. Mucosal Immuno12014; 7:718-29.

21. Blanchard C, Mingler M K, Vicario M, Abonia J P, Wu Y Y, Lu T X, et al. IL-13 involvement in eosinophilic esophagitis: transcriptome analysis and reversibility with glucocorticoids. J Allergy Clin Immunol 2007; 120:1292-300.

22. Harmon R M, Simpson C L, Johnson J L, Koetsier J L, Dubash A D, Najor N A, et al. Desmoglein-1/Erbin interaction suppresses ERK activation to support epidermal differentiation. J Clin Invest 2013; 123:1556-70.

23. Broussard J A, Getsios S, Green K J. Desmosome regulation and signaling in disease. Cell Tissue Res 2015; 360:501-12.

24. McDonald C, Chen F F, Ollendorff V, Ogura Y, Marchetto S, Lécine P, et al. A role for Erbin in the regulation of Nod2-dependent NF-kappaB signaling. J Biol Chem 2005; 280:40301-9.

25. Kufer T A, Kremmer E, Banks D J, Philpott D J. Role for erbin in bacterial activation of Nod2. Infect Immun 2006; 74:3115-24.

26. Boyden L M, Kam C Y, Hernández-Martin A, Zhou J, Craiglow B G, Sidbury R, et al. Dominant de novo DSP mutations cause erythrokeratodermia-cardiomyopathy syndrome. Hum Mol Genet 2015.

1. A method of treating an inflammatory skin disease associated with desmogelin-1 deficiency in a subject in need thereof comprising administering to the subject a therapeutically effective amount of an agent capable of restoring the expression of desmogelin-1.

2. A method of treating an inflammatory skin disease associated with desmogelin-1 deficiency in a subject in need thereof comprising administering to the subject a therapeutically effective amount of an inhibitor of NF-κB signaling pathway.

3. A method of treating an inflammatory skin disease associated with desmogelin-1 deficiency in a subject in need thereof comprising administering to the subject a therapeutically effective amount of an inhibitor of at least one cytokine selected from the group consisting of IL-6, IL-8, IL-1beta and TSLP.

4. The method of claim 1, wherein the inflammatory skin disease is selected from the group consisting of dermatitis, Netherton syndrome, SAM, and SAMEC syndromes.

5. The method of claim 1, which comprises, prior to the step of administering, a first step of determining whether the subject suffering from an inflammatory skin disease has a DSG1 deficiency.

6. The method of claim 5 wherein the first step comprises detecting the mutation that is responsible for the DSG1 deficiency.

7. The method of claim 6 wherein the mutation is selected from table A.

8. The method of claim 6 wherein the mutation is c.A1757C/p.H586P, or c.T1828C/p.S610P.

9. The method of claim 5 wherein the DSG1 deficiency is detected by determining the expression level of DSG1 in a sample obtained from the subject.

10. The method of claim 1 wherein the agent capable of restoring the expression of desmogelin-1 is a polynucleotide encoding for desmogelin 1.

11. The method of claim 10 wherein the polynucleotide comprises a nucleic acid sequence having at least 90% of identity with SEQ ID NO:1.

12. The method of claim 3 wherein the inhibitor is an antibody having specificity for IL-6, IL-8, IL-1beta or TSLP.

13. The method of 4, wherein the dermatitis is atopic dermatitis.

14. The method of claim 2, wherein the inflammatory skin disease is selected from the group consisting of dermatitis, Netherton syndrome, SAM, and SAMEC syndromes.

15. The method of claim 2, which comprises, prior to the step of administering, a first step of determining whether the subject suffering from an inflammatory skin disease has a DSG1 deficiency.

16. The method of claim 15 wherein the first step comprises detecting the mutation that is responsible for the DSG1 deficiency.

17. The method of claim 3, wherein the inflammatory skin disease is selected from the group consisting of dermatitis, Netherton syndrome, SAM, and SAMEC syndromes.

18. The method of claim 3, which comprises, prior to the step of administering, a first step of determining whether the subject suffering from an inflammatory skin disease has a DSG1 deficiency.

19. The method of claim 18 wherein the first step comprises detecting the mutation that is responsible for the DSG1 deficiency.
